(19) United States
(12) Patent Application Publication
(10) Pub. No.: US 2022/0386886 A1
(43) Pub. Date: Dec. 8, 2022

(54) NON-CONTACT HEART RHYTHM CATEGORY MONITORING SYSTEM AND METHOD

(71) Applicant: National Yang Ming Chiao Tung University, Hsinchu City (TW)

(72) Inventors: Bing-Fei WU, Hsinchu City (TW); Yin-Yin YANG, Taichung City (TW); Po-Wei HUANG, New Taipei City (TW); Bing-Jhang WU, Chiayi City (TW); Shao-En CHENG, Kaohsiung City (TW)

(21) Appl. No.: 17/452,239

(22) Filed: Oct. 26, 2021

(30) Foreign Application Priority Data
Jun. 2, 2021 (TW) 110120096

(51) Int. Cl. A61B 5/024; G06T 7/00; G06T 5/00; A61B 5/00; G16H 50/70; G16H 50/30; G16H 30/40

(52) U.S. Cl. CPC A61B 5/02416; G06T 7/0012; G06T 5/002; A61B 5/7267; A61B 5/7207; A61B 5/7278; A61B 5/7282; G16H 50/70; G16H 50/30; G16H 30/40; G06T 2207/30201; G06T 2207/20084; G06T 2207/30168

(57) ABSTRACT

The present disclosure provides a non-contact heart rhythm category monitoring system, which includes steps as follows. Facial images are continuously captured through an image sensor; images of a continuous target area for a predetermined duration are extracted from the facial images; non-contact physiological signal related to heartbeats are captured from the images of the continuous target area; the non-contact physiological signal are classified into a normal heart rhythm, an atrial fibrillation and a non-atrial fibrillation arrhythmia.

### RELATED APPLICATIONS

This application claims priority to Taiwan Patent Application No. 110120096, filed Jun. 2, 2021, the entirety of which is herein incorporated by reference.

### BACKGROUND

### Field of Invention

The present invention relates to systems and methods, and more particularly, non-contact heart rhythm category monitoring systems and methods.

### Description of Related Art

Heart rhythm refers to the frequency of heart contraction and beats and the number of beats per minute. Contact detection devices, such as a 24-hour ECG measuring instrument, a strap-type physiological signal measuring instrument, or a smart bracelet, can detect heart rhythm.

However, the contact detection device is relatively inconvenient to wear for the elderly, and is not suitable for long-term monitoring.

### SUMMARY

In one or more various aspects, the present disclosure is directed to non-contact heart rhythm category monitoring systems and methods.

An embodiment of the present disclosure is related to a non-contact heart rhythm category monitoring system. The non-contact heart rhythm category monitoring system includes an image sensor, a storage device and a processor. The image sensor is configured to continuously capture a plurality of facial images. The storage device is configured to store at least one instruction. The processor is coupled to the storage device, and the processor configured to access and execute the at least one instruction for: extracting images of a continuous target area from the facial images for a predetermined duration; obtaining a non-contact physiological signal related to heartbeats from the images of the continuous target area; classifying the non-contact physiological signal into a normal heart rhythm, an atrial fibrillation and a non-atrial fibrillation arrhythmia.

In one embodiment of the present disclosure, the processor accesses and executes the at least one instruction for: providing an option of whether to enable or disable a face detection; regulating a time length for a single sampling of the facial images; regulating another time length for each sampling interval for the facial images.

In one embodiment of the present disclosure, the processor accesses and executes the at least one instruction for: when the face detection is enabled, performing the face detection to correspondingly select the continuous target area.

In one embodiment of the present disclosure, the processor accesses and executes the at least one instruction for: when the face detection is disabled, extracting an entire frame of the facial images as the continuous target area.

In one embodiment of the present disclosure, the processor accesses and executes the at least one instruction for: converting pixel values of the continuous target area into the non-contact physiological signal related to the heartbeats through a signal model; enhancing the non-contact physiological signal to reduce a noise affection of at least one of ambient light and shadow, an artificial shaking, and a shaking of the image sensor; calculating at least one signal quality index of the non-contact physiological signal.

In one embodiment of the present disclosure, the processor accesses and executes the at least one instruction for: performing a spectrum analysis on the non-contact physiological signal to detect signal intensity values of a spectrum of the non-contact physiological signal at a plurality of frequencies, so that the at least one signal quality index includes the signal intensity values.

In one embodiment of the present disclosure, the processor accesses and executes the at least one instruction for: detecting a change of a standard deviation of a green pixel value in the non-contact physiological signal, so that the at least one signal quality index includes the change of the standard deviation of the green pixel value.

In one embodiment of the present disclosure, the processor accesses and executes the at least one instruction for: inputting the non-contact physiological signal into a deep convolutional neural network model to detect a waveform characteristic of a heart rhythm difference including a heart rhythm variability and a blood pulse volume, and to determine a preliminary heart rhythm category, wherein the deep convolutional neural network model is a deep network structure based on a filter size of a sample-level filter and a sample-level movement step length, so as to improve an accuracy of an automatic labeling of the non-contact physiological signal; setting a total recording period of a combination of continuous samplings of the non-contact physiological signal according to a target duration, and performing a voting mechanism on the preliminary heart rhythm category to determine a final heart rhythm category, and the final heart rhythm category distinguishes the normal heart rhythm, the atrial fibrillation and the non-atrial fibrillation arrhythmia.

In one embodiment of the present disclosure, the processor accesses and executes the at least one instruction for: when the target duration is not set by a user, evaluating at least one signal quality index in different time lengths to automatically set the target duration.

In one embodiment of the present disclosure, the processor accesses and executes the at least one instruction for: accepting a user setting to determine the target duration.

Another embodiment of the present disclosure is related to a non-contact heart rhythm category monitoring method. The non-contact heart rhythm category monitoring method includes steps of: continuously capturing a plurality of facial images through an image sensor; extracting images of a continuous target area from the facial images for a predetermined duration; obtaining a non-contact physiological signal related to heartbeats from the images of the continuous target area; classifying the non-contact physiological signal into a normal heart rhythm, an atrial fibrillation and a non-atrial fibrillation arrhythmia.

In one embodiment of the present disclosure, the non-contact heart rhythm category monitoring method further includes steps of: providing an option of whether to enable or disable a face detection; regulating a time length for a single sampling of the facial images; regulating another time length for each sampling interval for the facial images.

In one embodiment of the present disclosure, the non-contact heart rhythm category monitoring method further includes steps of: when the face detection is enabled, performing the face detection to correspondingly select the continuous target area.

In one embodiment of the present disclosure, the non-contact heart rhythm category monitoring method further includes steps of: when the face detection is disabled, extracting an entire frame of the facial images as the continuous target area.

In one embodiment of the present disclosure, the step of obtaining a non-contact physiological signal related to heartbeats from the images of the continuous target area includes: converting pixel values of the continuous target area into the non-contact physiological signal related to the heartbeats through a signal model; enhancing the non-contact physiological signal to reduce a noise affection of at least one of ambient light and shadow, an artificial shaking, and a shaking of the image sensor; calculating at least one signal quality index of the non-contact physiological signal.

In one embodiment of the present disclosure, the step of calculating the at least one signal quality index of the non-contact physiological signal includes: performing a spectrum analysis on the non-contact physiological signal to detect signal intensity values of a spectrum of the non-contact physiological signal at a plurality of frequencies, so that the at least one signal quality index includes the signal intensity values.

In one embodiment of the present disclosure, the step of calculating the at least one signal quality index of the non-contact physiological signal includes: detecting a change of a standard deviation of a green pixel value in the non-contact physiological signal, so that the at least one signal quality index includes the change of the standard deviation of the green pixel value.

In one embodiment of the present disclosure, the step of classifying the non-contact physiological signal into the normal heart rhythm, the atrial fibrillation and the non-atrial fibrillation arrhythmia includes: inputting the non-contact physiological signal into a deep convolutional neural network model to detect a waveform characteristic of a heart rhythm difference including a heart rhythm variability and a blood pulse volume, and to determine a preliminary heart rhythm category, wherein the deep convolutional neural network model is a deep network structure based on a filter size of a sample-level filter and a sample-level movement step length, so as to improve an accuracy of an automatic labeling of the non-contact physiological signal; setting a total recording period of a combination of continuous samplings of the non-contact physiological signal according to a target duration, and performing a voting mechanism on the preliminary heart rhythm category to determine a final heart rhythm category, and the final heart rhythm category distinguishes the normal heart rhythm, the atrial fibrillation and the non-atrial fibrillation arrhythmia.

In one embodiment of the present disclosure, the step of classifying the non-contact physiological signal into the normal heart rhythm, the atrial fibrillation and the non-atrial fibrillation arrhythmia further includes: when the target duration is not set by a user, evaluating at least one signal quality index in different time lengths to automatically set the target duration.

In one embodiment of the present disclosure, the step of classifying the non-contact physiological signal into the normal heart rhythm, the atrial fibrillation and the non-atrial fibrillation arrhythmia further includes: accepting a user setting to determine the target duration.

Many of the attendant features will be more readily appreciated, as the same becomes better understood by reference to the following detailed description considered in connection with the accompanying drawings.

### BRIEF DESCRIPTION OF THE DRAWINGS

The invention can be more fully understood by reading the following detailed description of the embodiment, with reference made to the accompanying drawings as follows:

FIG. 1A is a architecture diagram of a non-contact heart rhythm category monitoring system according to one embodiment of the present disclosure;

FIG. 1B is a block diagram of the non-contact heart rhythm category monitoring system according to one embodiment of the present disclosure;

FIG. 2 is a flow chart of non-contact heart rhythm category monitoring method according to one embodiment of the present disclosure;

FIG. 3 is a block diagram of a sampling module according to one embodiment of the present disclosure;

FIG. 4 is a block diagram of a physiological signal calculation module according to one embodiment of the present disclosure;

FIG. 5 is a block diagram of a heart rhythm classification module according to one embodiment of the present disclosure;

FIG. 6 is a flow chart of a step of FIG. 2 according to one embodiment of the present disclosure;

FIG. 7A is a schematic diagram of a facial image according to one embodiment of the present disclosure;

FIG. 7B is a schematic diagram of detecting the coordinates of facial feature points in the facial image of FIG. 7A; and

FIG. 7C is a target area selected from the facial feature points of FIG. 7B.

### DETAILED DESCRIPTION

Reference will now be made in detail to the present embodiments of the invention, examples of which are illustrated in the accompanying drawings. Wherever possible, the same reference numbers are used in the drawings and the description to refer to the same or like parts.

As used in the description herein and throughout the claims that follow, the meaning of “a”, “an”, and “the” includes reference to the plural unless the context clearly dictates otherwise. Also, as used in the description herein and throughout the claims that follow, the terms “comprise or comprising”, “include or including”, “have or having”, “contain or containing” and the like are to be understood to be open-ended, i.e., to mean including but not limited to. As used in the description herein and throughout the claims that follow, the meaning of “in” includes “in” and “on” unless the context clearly dictates otherwise.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of the embodiments. As used herein, the term “and/or” includes any and all combinations of one or more of the associated listed items.

Unless otherwise defined, all terms (including technical and scientific terms) used herein have the same meaning as commonly understood by one of ordinary skill in the art to which example embodiments belong. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

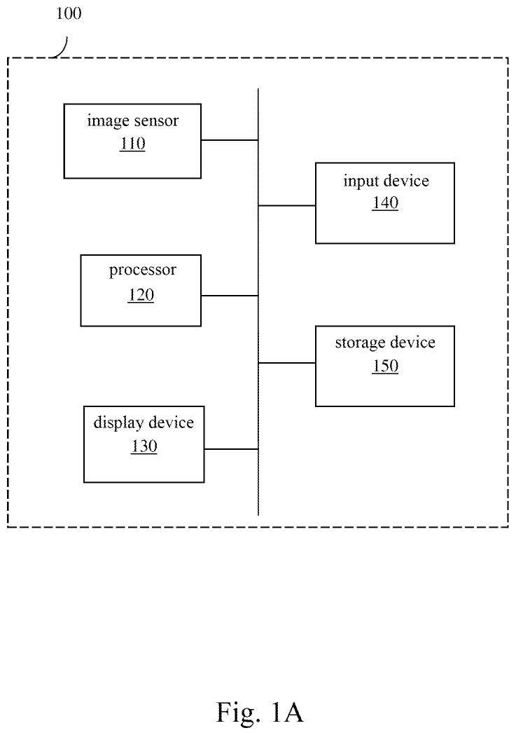

Referring to FIG. 1A and FIG. 1B, in one aspect, the present disclosure is directed to a non-contact heart rhythm category monitoring system 100. The non-contact heart rhythm category monitoring system 100 may be easily integrated into a computer and may be applicable or readily adaptable to all technologies. Technical advantages are generally achieved by the non-contact heart rhythm category monitoring system 100 according to embodiments of the present disclosure. Herewith the Non-contact heart rhythm category monitoring system 100 is described below with FIG. 1A and FIG. 1B.

The subject disclosure provides the non-contact heart rhythm category monitoring system 100 in accordance with the subject technology. Various aspects of the present technology are described with reference to the drawings. In the following description, for purposes of explanation, numerous specific details are set forth in order to provide a thorough understanding of one or more aspects. It can be evident, however, that the present technology can be practiced without these specific details. In other instances, well-known structures and devices are shown in block diagram form in order to facilitate describing these aspects. The word “exemplary” is used herein to mean “serving as an example, instance, or illustration.” Any embodiment described herein as “exemplary” is not necessarily to be construed as preferred or advantageous over other embodiments.

FIG. 1A is a block diagram of the non-contact heart rhythm category monitoring system 100 according to one embodiment of the present disclosure. As shown in FIG. 1A, the non-contact heart rhythm category monitoring system 100 includes an image sensor 110, a processor 120, a display device 130, an input device 140 and a storage device 150. For example, the storage device 150 can be a hard drive, a flash memory or another storage device, the processor 120 can be a central processing unit, and a display device 130 can be a built-in the display screen or an external screen.

In structure, the processor 120 is coupled to the image sensor 110, the display device 130, the input device 140 and the storage device 150. It should be noted that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present. For example, the image sensor 110 may be a built-in image sensor that is directly connected to the processor 120, or the image sensor 110 may be an external image sensor that is indirectly connected to the processor 120 through the connection circuit.

In one embodiment of the present disclosure, the image sensor 110 is configured to continuously capture a plurality of facial images, the storage device 150 store at least one instruction, the processor 120 is coupled to the storage device 150, and the processor 120 accesses and executes the at least one instruction for: extracting images of a continuous target area from the facial images for a predetermined duration; obtaining a non-contact physiological signal related to heartbeats from the images of the continuous target area; classifying the non-contact physiological signal into a normal heart rhythm, an atrial fibrillation and a non-atrial fibrillation arrhythmia. For example, the above-mentioned image can be image data without presenting a visualized image, thereby increasing the speed of calculation and circumventing the problem of personal privacy.

In one embodiment of the present disclosure, the processor 120 accesses and executes the at least one instruction for: providing an option of whether to enable or disable a face detection; regulating a time length for a single sampling of the facial images; regulating another time length for each sampling interval for the facial images. For example, the display device 130 can render the option of whether to enable or disable the face detection, and the user can select whether to enable the face detection through the input device 140.

In one embodiment of the present disclosure, the processor 120 accesses and executes the at least one instruction for: when the face detection is enabled, performing the face detection to correspondingly select the continuous target area.

In one embodiment of the present disclosure, the processor 120 accesses and executes the at least one instruction for: when the face detection is disabled, extracting an entire frame of the facial images as the continuous target area.

In one embodiment of the present disclosure, the processor 120 accesses and executes the at least one instruction for: converting pixel values of the continuous target area into the non-contact physiological signal related to the heartbeats through a signal model; enhancing the non-contact physiological signal to reduce a noise affection of at least one of ambient light and shadow, an artificial shaking, and a shaking of the image sensor; calculating at least one signal quality index of the non-contact physiological signal.

In one embodiment of the present disclosure, the processor 120 accesses and executes the at least one instruction for: performing a spectrum analysis on the non-contact physiological signal to detect signal intensity values of a spectrum of the non-contact physiological signal at a plurality of frequencies, so that the at least one signal quality index includes the signal intensity values.

In one embodiment of the present disclosure, the processor 120 accesses and executes the at least one instruction for: detecting a change of a standard deviation of a green pixel value in the non-contact physiological signal, so that the at least one signal quality index includes the change of the standard deviation of the green pixel value. For example, compared to other colors, the quality represented by the green pixel value is more reliable.

In one embodiment of the present disclosure, the processor 120 accesses and executes the at least one instruction for: inputting the non-contact physiological signal into a deep convolutional neural network model to detect a waveform characteristic of a heart rhythm difference including a heart rhythm variability and a blood pulse volume, and to determine a preliminary heart rhythm category, wherein the deep convolutional neural network model is a deep network structure based on a filter size of a sample-level filter and a sample-level movement step length, so as to improve an accuracy of an automatic labeling of the non-contact physiological signal; setting a total recording period of a combination of continuous samplings of the non-contact physiological signal according to a target duration, and performing a voting mechanism on the preliminary heart rhythm category to determine a final heart rhythm category, and the final heart rhythm category distinguishes the normal heart rhythm, the atrial fibrillation and the non-atrial fibrillation arrhythmia. For example, the target duration may be approximately equal to or less than the predetermined duration as mentioned above, but the present disclosure is not limited thereto, and those skilled in the art should flexibly adjust the duration depending on the actual application.

In one embodiment of the present disclosure, the processor 120 accesses and executes the at least one instruction for: when the target duration is not set by a user, evaluating at least one signal quality index in different time lengths to automatically set the target duration.

In one embodiment of the present disclosure, the processor 120 accesses and executes the at least one instruction for: accepting a user setting to determine the target duration. For example, the user can set the target duration through the input device 140.

It should be noted that the storage device 150 store at least one instruction, and the processor 120 accesses and executes the at least one instruction to perform functions, procedures, processing, etc., which can be represented by modules and units below.

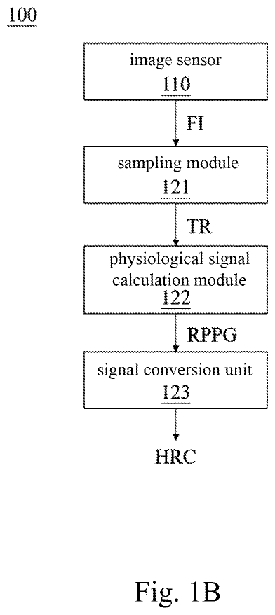

Referring to FIG. 1B and FIG. 2, FIG. 1B is a block diagram of the non-contact heart rhythm category monitoring system 100 according to one embodiment of the present disclosure, and FIG. 2 is a flowchart of a non-contact heart rhythm category monitoring method M100 according to one embodiment of the present disclosure. For example, the non-contact heart rhythm category monitoring system 100 can be a non-contact atrial fibrillation and other heart rhythm category monitoring system, and the non-contact heart rhythm category monitoring method M100 can be a non-contact atrial fibrillation and other heart rhythm category monitoring method.

In this embodiment, the non-contact heart rhythm category monitoring system 100 can be used to execute the non-contact heart rhythm category monitoring method M100 for non-contact the atrial fibrillation and other the heart rhythm category monitoring, where the non-contact heart rhythm category monitoring system includes the image sensor 110, a sampling module 121, a physiological signal calculation module 122, and a heart rhythm classification module 123, and the non-contact heart rhythm category monitoring method M100 includes steps S101 to S118.

The image sensor 110 is used to continuously capture a plurality of images.

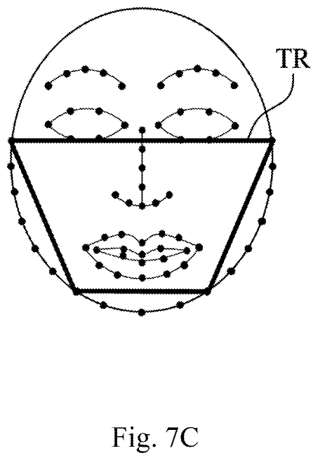

Referring to FIG. 2, in step S101, the image sensor 110 can continuously capture a plurality of images; specifically, referring to FIG. 7A, which is a schematic diagram of the overall image FI of the captured entire human face according to an embodiment of the present disclosure.

In one embodiment, the image sensor 110 may be an optical sensing element or a camera unit, a video camera, or a video recorder.

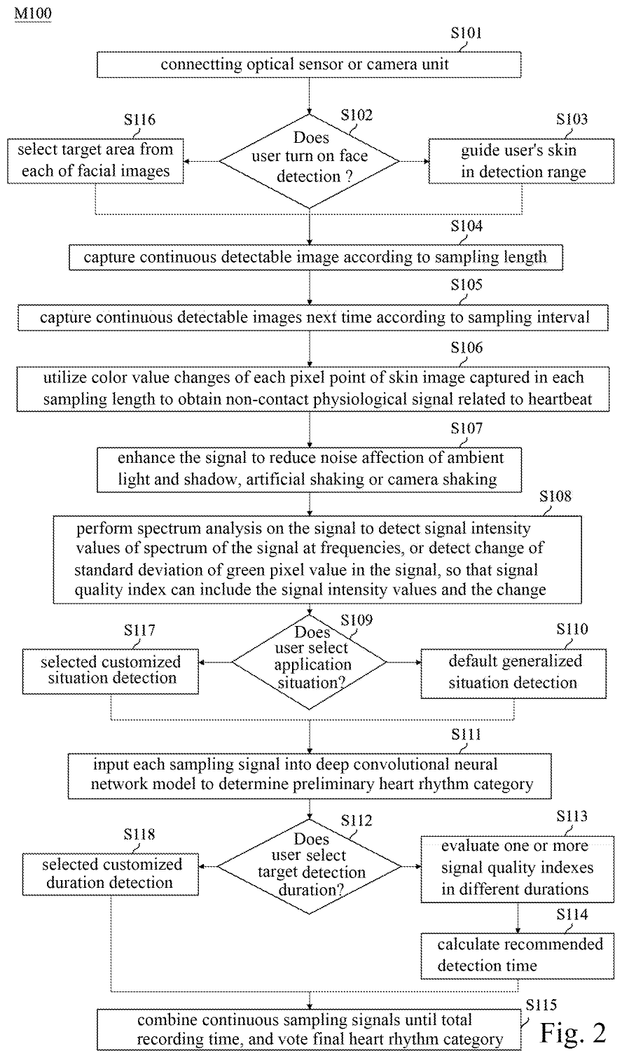

Referring to FIG. 3, which shows a block diagram of the sampling module 121 according to one embodiment of the present disclosure. The sampling module 121 includes a target area selection unit 121a, a sampling length control unit 121b, and a sampling interval control unit 121c. The sampling module 121 captures images of the continuous target area for the predetermined duration. The target area selection unit 121a provides a user interface having an option of whether to enable or disable the face detection. The sampling length control unit 121b regulates a time length for a single sampling of the continuous images, and the sampling interval control unit 121c another time length for each sampling interval for the images. In one embodiment, the target area selection unit 121a, the sampling length control unit 121b, and the sampling interval control unit 121c can be selectively executed separately, simultaneously or in pairs according to actual application conditions.

Referring to FIG. 2, in step S102, the target area selection unit 121a can select whether to turn on the face detection.

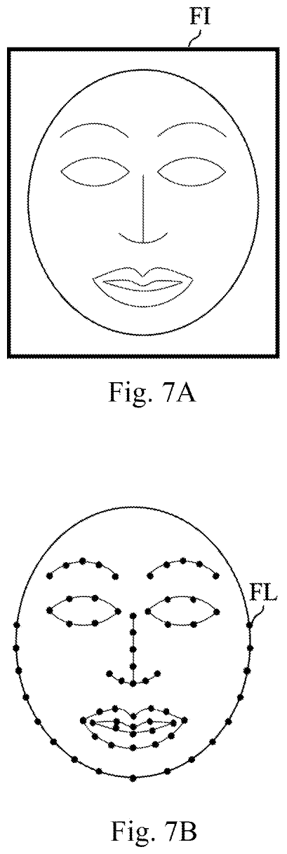

Referring to FIG. 6, FIG. 7A, FIG. 7B, and FIG. 7C together, in step S103, the system can ask the user to be relatively fixed at a detectable distance from the image sensor, and the overall image FI in FIG. 7A is used as the target area TR.

In step S116, the image sensor 110 first captures the overall image FI of the user, and uses face feature extraction technology to capture the facial feature points FL in the overall image FI, where the face feature extraction technology can be a Dlib toolkit that includes machine learning algorithms and tools; however, the face detection method is not limited thereto. FIG. 7B is a schematic diagram of detecting the coordinates of facial feature points FL in the facial image FI of FIG. 7A.

Furthermore, the target area TR with better facial image quality is selected from the detected facial feature points FL, and FIG. 7C is a schematic diagram of the target area TR that is a frame selected from the facial feature points FL of FIG. 7B, but the frame selection method is not limited thereto.

According to some embodiments of the present disclosure, the non-contact heart rhythm category monitoring system can detect in more than one target area.

In step S104, the sampling length control unit 121b captures a plurality of continuous detectable image data according to a configurable sampling length, or captures a plurality of continuous detectable image data according to a sampling length preset by the system.

In step S105, the sampling interval control unit 121c captures a plurality of continuous detectable image data in the next time according to the configurable sampling interval, or captures a plurality of continuous detectable image data in the next time according to the sampling interval preset by the system.

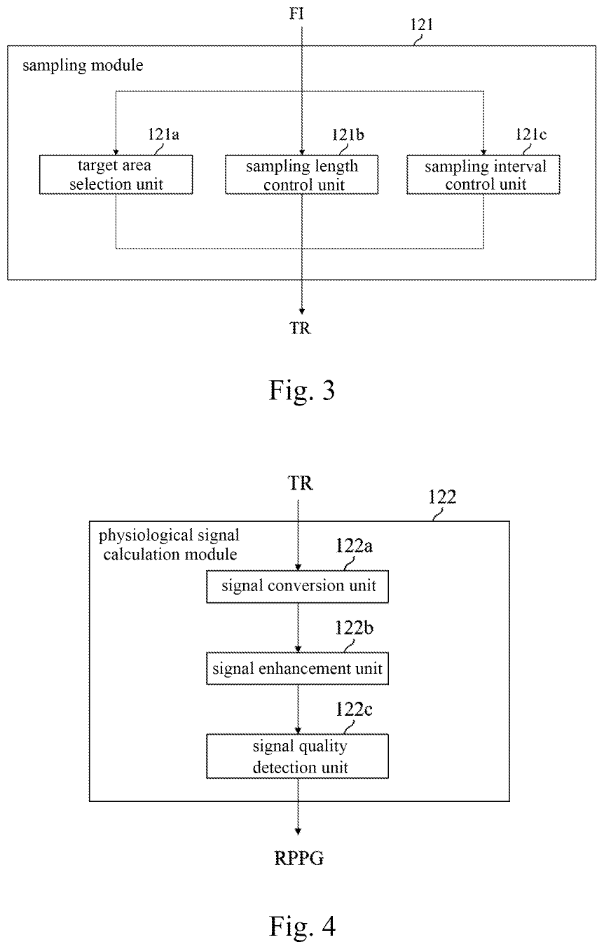

Referring to FIG. 4, which is a block diagram of the physiological signal calculation module 122 according to one embodiment of the present disclosure. The physiological signal calculation module 122 includes a signal conversion unit 122a, a signal enhancement unit 122b, and a signal quality detection unit 122c.

The physiological signal calculation module 122 is used to obtain the non-contact physiological signals related to heartbeats.

According to some embodiments of the present disclosure, in step S106 to step S108, the physiological signal calculation module 122 uses the signal conversion unit 122a to convert pixel values of the continuous target area into the non-contact physiological signal related to the heartbeats through a signal model. The signal enhancement unit 122b is used to enhance the non-contact physiological signal to reduce a noise affection of at least one of ambient light and shadow, an artificial shaking, and a camera shaking. The signal quality detection unit 122c is used to calculate one or more signal quality indexes of the non-contact physiological signal. The signal quality detection unit 122c can perform a spectrum analysis on the non-contact physiological signal to detect signal intensity values of a spectrum of the non-contact physiological signal at a plurality of frequencies, and/or can detect a change of a standard deviation of a green pixel value in the non-contact physiological signal, so that the signal quality index can include the signal intensity values and/or the change of the standard deviation of the green pixel value.

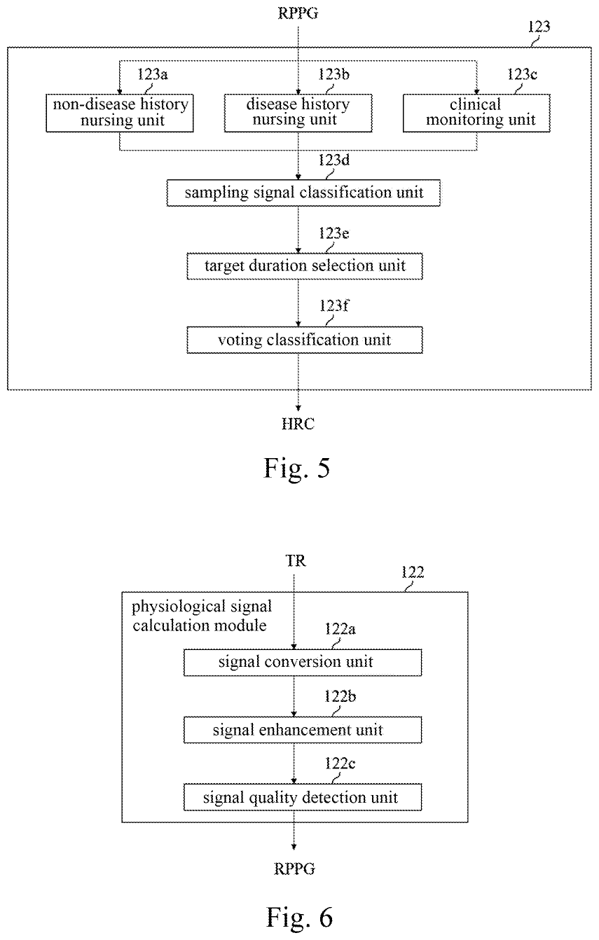

Referring to FIG. 5, FIG. 5 is a block diagram of the heart rhythm classification module 123 according to one embodiment of the present disclosure. The heart rhythm classification module 123 includes a non-disease history nursing unit 123a, a disease history nursing unit 123b, a clinical monitoring unit 123c, a sampling signal classification unit 123d, a target duration selection unit 123e, and a voting classification unit 123f.

The heart rhythm classification module 123 is used to perform the atrial fibrillation on the non-contact physiological signals to classify the normal heart rhythm and other heart rhythms of non-atrial fibrillation.

According to some embodiments of the present disclosure, in step S109, an interface is provided for the user to select the non-disease history nursing unit 123a, the disease history nursing unit 123b, and the clinical monitoring unit 123c. The non-disease history nursing unit 123a is used to distinguish the atrial fibrillation from the normal heart rhythm. The disease history nursing unit 123b is used to distinguish the atrial fibrillation from other heart rhythms that include the normal heart rhythm and other heart rhythms of the non-atrial fibrillation. The clinical monitoring unit 123c is used to distinguish the atrial fibrillation from other heart rhythms of the non-atrial fibrillation.

In step S110, in one embodiment, there is no need to select the use situation, and the system uses the disease history nursing unit 123b as the default detection model, and this description is the same as that of step S109 and is not be repeated herein.

According to some embodiments of the present disclosure, in step S117, the non-disease history nursing unit 123a, the disease history nursing unit 123b, and the clinical monitoring unit 123c can be selectively and separately executed according to actual application.

In step S111, the sampling signal classification unit 123d is used to input the non-contact physiological signal into a deep convolutional neural network model to detect a waveform characteristic of a heart rhythm difference including a heart rhythm variability and a blood pulse volume, and to determine a preliminary heart rhythm category, wherein the deep convolutional neural network model is a deep network structure based on a filter size of a sample-level filter and a sample-level movement step length, so as to improve an accuracy of an automatic labeling of the non-contact physiological signal, but the heart rhythm classification method is not limited thereto.

In step S112, the target duration selection unit 123e is used to provide a user with a selection of the detection duration, and use one or multiple signal quality indexes within different durations (time lengths) to a result of the heart rhythm category in a period with more confidences, where the detection duration can be adjusted by the user in step S118.

In another embodiment, the target duration selection unit 123 in step S113 may also automatically evaluate one or more signal quality indexes of the sampling signal used for the duration by the system, and then in step S114, the system calculates the recommended detection duration for the user.

In step S115, the voting classification unit 123f is used to combine the continuous sampling signals until the total recording time set by the target duration, and the voting mechanism determines the final heart rhythm category.

According to some embodiments of the present disclosure, the non-contact heart rhythm category monitoring system can perform detection in one or more lengths of time.

According to some embodiments of the present disclosure, the non-contact heart rhythm category monitoring system can detect the atrial fibrillation and distinguish it from the normal heart rhythm.

According to some embodiments of the present disclosure, the non-contact heart rhythm category monitoring system can detect the atrial fibrillation and distinguish it from other heart rhythms of the non-atrial fibrillation.

According to some embodiments of the present disclosure, the non-contact heart rhythm category monitoring system can detect the atrial fibrillation and distinguish it from other arrhythmia categories that include the normal heart rhythm and the non-the atrial fibrillation.

In view of above, according to the various embodiments of the present disclosure, the purpose of monitoring the atrial fibrillation in other heart rhythms including the normal heart rhythm and non-the atrial fibrillation can be achieved. Furthermore, through the sampling module 121, the physiological signal calculation module 122 and the heart rhythm classification module 123, the atrial fibrillation detection output is more accurate.

It should be noted that in the non-contact heart rhythm category monitoring system 100, the image sensor 110, the sampling module 121, the target area selection unit 121a, the sampling length control unit 121b, the feature point coordinate detection unit 121a, the target area frame selection unit 121b, the sampling interval control unit 121c, the physiological signal calculation module 122, the signal conversion unit 122a, the signal enhancement unit 122b, the signal quality detection unit 122c, the heart rhythm classification module 123, the non-disease history nursing unit 123a, the disease history nursing unit 123b, the clinical monitoring unit 123c, the sampling signal classification unit 123d, the target duration selection unit 123e, the voting classification unit 123f can be implemented with hardware, software, firmware or the combination thereof.

In a control experiment, as to a physiological arrhythmia measuring device, such as a 24-hour electrocardiogram or a smart watch, there is discomfort caused by contact wearing, especially for the atrial fibrillation high-risk group, mostly elderly people groups, and therefore the acceptance of this contact type equipment is generally not high. Compared with the control experiment, the present disclosure uses non-contact image input as the measurement method to monitor the user's physical conditions without adversely affecting the user.

In a control experiment, the correlation with the main heartbeat period is used as a feature, and its peak selection tool and the selected signal quality easily affect the accuracy of detecting the atrial fibrillation. Compared with the control experiment, the present disclosure uses the training method to improve the effect of peak selection, and extract more features related to the atrial fibrillation, so as to improve the detection accuracy.

In a control experiment, only the category of the normal heart rhythm is considered as the control group of the atrial fibrillation, so the classifier distinguishes the atrial fibrillation from the normal heart rhythm only. However, in addition to the atrial fibrillation and the normal heart rhythm, the actual heart rhythm types cover other arrhythmia types of the non-atrial fibrillation. Compared with the control experiment, the present disclosure provides multiple scenarios (e.g., normal the heart rhythm category, other arrhythmia categories of the non-atrial fibrillation, and a comprehensive category that includes the normal heart rhythm and other arrhythmia categories of the non-atrial fibrillation) for the market needs.

In practice, for example, the present disclosure proposes a training framework for the image-based atrial fibrillation detection that can distinguish multiple the heart rhythm categories. The training framework can distinguish the atrial fibrillation from other the heart rhythm categories, such as the normal heart rhythm, another arrhythmia category of the non-atrial fibrillation, and yet another arrhythmia category including the normal heart rhythm and the non-atrial fibrillation.

In practice, for example, the present disclosure proposes multi-featured waveform recognition learning, using the arrhythmia of the atrial fibrillation and other physiological characteristics to enhance the recognition of the atrial fibrillation, complementing the current lack of uniqueness in the atrial fibrillation detection research, avoiding the overlapping of characteristics of other arrhythmias of the non-atrial fibrillation, and can improve the detection accuracy of all situational tasks.

In practice, for example, the present disclosure uses overlapping sampling to avoid missing related heart rhythm detection features in the signal acquisition interval, to increase the amount of data at the same time, and to increase the accuracy of heart rhythm differentiation.

In practice, for example, the present disclosure uses the shortening of the sampling time and the voting mechanism to adjust the structure of the heart rhythm judgment within a fixed time, reducing the probability of misjudgment caused by the loss of signal quality or characteristics, so that the system can output heart rhythm judgment accurately.

In practice, for example, the present disclosure, using signal confidence enhancement can use an unmanned face detection system that can directly capture screen images and use the enhanced image physiological signal unit to smoothly execute the heart rhythm detection unit.

It will be apparent to those skilled in the art that various modifications and variations can be made to the structure of the present invention without departing from the scope or spirit of the invention. In view of the foregoing, it is intended that the present invention cover modifications and variations of this invention provided they fall within the scope of the following claims.

What is claimed is:

1. A non-contact heart rhythm category monitoring system, comprising:
an image sensor configured to continuously capture a plurality of facial images;
a storage device configured to store at least one instruction; and
a processor coupled to the storage device, and the processor configured to access and execute the at least one instruction for:
extracting images of a continuous target area from the facial images for a predetermined duration;
obtaining a non-contact physiological signal related to heartbeats from the images of the continuous target area; and
classifying the non-contact physiological signal into a normal heart rhythm, an atrial fibrillation and a non-atrial fibrillation arrhythmia.

2. The non-contact heart rhythm category monitoring system of claim 1, wherein the processor accesses and executes the at least one instruction for:
providing an option of whether to enable or disable a face detection;
regulating a time length for a single sampling of the facial images; and
regulating another time length for each sampling interval for the facial images.

3. The non-contact heart rhythm category monitoring system of claim 1, wherein the processor accesses and executes the at least one instruction for:
when the face detection is enabled, performing the face detection to correspondingly select the continuous target area.

4. The non-contact heart rhythm category monitoring system of claim 2, wherein the processor accesses and executes the at least one instruction for:
when the face detection is disabled, extracting an entire frame of the facial images as the continuous target area.

5. The non-contact heart rhythm category monitoring system of claim 2, wherein the processor accesses and executes the at least one instruction for:
converting pixel values of the continuous target area into the non-contact physiological signal related to the heartbeats through a signal model;
enhancing the non-contact physiological signal to reduce a noise affection of at least one of ambient light and shadow, an artificial shaking, and a shaking of the image sensor; and
calculating at least one signal quality index of the non-contact physiological signal.

6. The non-contact heart rhythm category monitoring system of claim 5, wherein the processor accesses and executes the at least one instruction for:
performing a spectrum analysis on the non-contact physiological signal to detect signal intensity values of a spectrum of the non-contact physiological signal at a plurality of frequencies, so that the at least one signal quality index includes the signal intensity values.

7. The non-contact heart rhythm category monitoring system of claim 5, wherein the processor accesses and executes the at least one instruction for:
detecting a change of a standard deviation of a green pixel value in the non-contact physiological signal, so that the at least one signal quality index includes the change of the standard deviation of the green pixel value.

8. The non-contact heart rhythm category monitoring system of claim 5, wherein the processor accesses and executes the at least one instruction for:
inputting the non-contact physiological signal into a deep convolutional neural network model to detect a waveform characteristic of a heart rhythm difference including a heart rhythm variability and a blood pulse volume, and to determine a preliminary heart rhythm category, wherein the deep convolutional neural network model is a deep network structure based on a filter size of a sample-level filter and a sample-level movement step length, so as to improve an accuracy of an automatic labeling of the non-contact physiological signal; and
setting a total recording period of a combination of continuous samplings of the non-contact physiological signal according to a target duration, and performing a voting mechanism on the preliminary heart rhythm category to determine a final heart rhythm category, and the final heart rhythm category distinguishes the normal heart rhythm, the atrial fibrillation and the non-atrial fibrillation arrhythmia.

9. The non-contact heart rhythm category monitoring system of claim 8, wherein the processor accesses and executes the at least one instruction for:
when the target duration is not set by a user, evaluating at least one signal quality index in different time lengths to automatically set the target duration.

10. The non-contact heart rhythm category monitoring system of claim 8, wherein the processor accesses and executes the at least one instruction for:
accepting a user setting to determine the target duration.

11. A non-contact heart rhythm category monitoring method, comprising steps of:
continuously capturing a plurality of facial images through an image sensor;
extracting images of a continuous target area from the facial images for a predetermined duration;
obtaining a non-contact physiological signal related to heartbeats from the images of the continuous target area; and
classifying the non-contact physiological signal into a normal heart rhythm, an atrial fibrillation and a non-atrial fibrillation arrhythmia.

12. The non-contact heart rhythm category monitoring method of claim 11, further comprising:
providing an option of whether to enable or disable a face detection;
regulating a time length for a single sampling of the facial images; and
regulating another time length for each sampling interval for the facial images.

13. The non-contact heart rhythm category monitoring method of claim 12, further comprising:
when the face detection is enabled, performing the face detection to correspondingly select the continuous target area.

14. The non-contact heart rhythm category monitoring method of claim 12, further comprising:
when the face detection is disabled, extracting an entire frame of the facial images as the continuous target area.

15. The non-contact heart rhythm category monitoring method of claim 12, wherein the step of obtaining a non-contact physiological signal related to heartbeats from the images of the continuous target area comprises:
converting pixel values of the continuous target area into the non-contact physiological signal related to the heartbeats through a signal model;
enhancing the non-contact physiological signal to reduce a noise affection of at least one of ambient light and shadow, an artificial shaking, and a shaking of the image sensor; and
calculating at least one signal quality index of the non-contact physiological signal.

16. The non-contact heart rhythm category monitoring method of claim 15, wherein the step of calculating the at least one signal quality index of the non-contact physiological signal comprises:
performing a spectrum analysis on the non-contact physiological signal to detect signal intensity values of a spectrum of the non-contact physiological signal at a plurality of frequencies, so that the at least one signal quality index includes the signal intensity values.

17. The non-contact heart rhythm category monitoring method of claim 15, wherein the step of calculating the at least one signal quality index of the non-contact physiological signal comprises:
detecting a change of a standard deviation of a green pixel value in the non-contact physiological signal, so that the at least one signal quality index includes the change of the standard deviation of the green pixel value.

18. The non-contact heart rhythm category monitoring method of claim 15, wherein the step of classifying the non-contact physiological signal into the normal heart rhythm, the atrial fibrillation and the non-atrial fibrillation arrhythmia comprises:
inputting the non-contact physiological signal into a deep convolutional neural network model to detect a waveform characteristic of a heart rhythm difference including a heart rhythm variability and a blood pulse volume, and to determine a preliminary heart rhythm category, wherein the deep convolutional neural network model is a deep network structure based on a filter size of a sample-level filter and a sample-level movement step length, so as to improve an accuracy of an automatic labeling of the non-contact physiological signal; and
setting a total recording period of a combination of continuous samplings of the non-contact physiological signal according to a target duration, and performing a voting mechanism on the preliminary heart rhythm category to determine a final heart rhythm category, and the final heart rhythm category distinguishes the normal heart rhythm, the atrial fibrillation and the non-atrial fibrillation arrhythmia.

19. The non-contact heart rhythm category monitoring method of claim 18, wherein the step of classifying the non-contact physiological signal into the normal heart rhythm, the atrial fibrillation and the non-atrial fibrillation arrhythmia further comprises:
when the target duration is not set by a user, evaluating at least one signal quality index in different time lengths to automatically set the target duration.

20. The non-contact heart rhythm category monitoring method of claim 18, wherein the step of classifying the non-contact physiological signal into the normal heart rhythm, the atrial fibrillation and the non-atrial fibrillation arrhythmia further comprises:
accepting a user setting to determine the target duration.
